(19) United States
(12) Patent Application Publication
(10) Pub. No.: US 2015/0112227 A1
(43) Pub. Date: Apr. 23, 2015

(54) PHLEBOTOMY AID DEVICE

(71) Applicants: Leonard DiGiovanna, West Hempstead, NY (US); Philip D. Ragno, Glen Head, NY (US)

(72) Inventors: Leonard DiGiovanna, West Hempstead, NY (US); Philip D. Ragno, Glen Head, NY (US)

(21) Appl. No.: 14/327,606

(22) Filed: Jul. 10, 2014

Related U.S. Application Data
(60) Provisional application No. 61/893,026, filed on Oct. 18, 2013.

(51) Int. Cl. A61B 5/15

(52) U.S. Cl. CPC A61B 5/150068
USPC 600/573

(57) ABSTRACT

A phlebotomy aid device may include at least two pads each having a body side. An adhesive may attach to the body side of the at least two pads. The at least two pads may be connected by a connecting mechanism. A tightening mechanism may be attached to the connecting mechanism. The device may be attached to skin on sides of a vein of a patient. The tightening mechanism may allow the connecting mechanism to pull on the at least two pads, which in turn, may pull on the skin attached to the at least two pads. The device may allow for the stretching of skin in order to immobilize veins while not encroaching on a sterile field.

### CROSS-REFERENCE TO RELATED APPLICATION

This application claims the benefit of U.S. provisional application No. 61/893,026, filed Oct. 18, 2013, the contents of which are herein incorporated by reference.

### BACKGROUND OF THE INVENTION

The present invention relates to phlebotomy aid devices and, more particularly, to a phlebotomy aid device that does not encroach on a sterile field.

Currently, poor veins and flaccid or expanded skin presents a problem for phlebotomists and health care givers when trying to access veins for blood drawing or intravenous setup. Other devices have been designed to immobilize a specific vein and end up obstructing the phlebotomist's path to the vein. Other devices are more cumbersome to use and require a higher level of expertise.

As can be seen, there is a need for a phlebotomy aid device that may allow for the stretching of skin in order to immobilize veins while not encroaching on the sterile field.

### SUMMARY OF THE INVENTION

In one aspect of the present invention, a phlebotomy aid device comprises: at least two pads each having a body side; an adhesive attached to the body side of the at least two pads; and a connecting mechanism that connects to the at least two pads.

In another aspect of the present invention, a method for preparing a vein of a patient for a phlebotomy procedure comprises: attaching an adhesive attached to a body side of at least two pads to a section of skin of the patient, wherein the at least two pads are positioned on either side of the vein, wherein the at least two pads are connected to a connecting mechanism; and tightening the connecting mechanism, wherein the connecting mechanism then pulls on the at least two pads, wherein the at least two pads pull on the skin providing a taut surface.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention shown in use;

FIG. 2 is a perspective view of an exemplary embodiment of the present invention;

FIG. 3 is a detailed section view of an exemplary embodiment of the present invention taken along line 3-3- in FIG. 1;

FIG. 4 is a perspective view of an alternate embodiment of the present invention;

FIG. 5 is a perspective view of an alternate embodiment of the present invention;

FIG. 6 is a perspective view of an alternate embodiment of the present invention;

FIG. 7 is a perspective view of an alternate embodiment of the present invention; and

FIG. 8 is a perspective view of an alternate embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a phlebotomy aid device that may include at least two pads each having a body side. An adhesive may attach to the body side of the at least two pads. The at least two pads may be connected by a connecting mechanism. A tightening mechanism may be attached to the connecting mechanism. The device may be attached to skin on sides of a vein of a patient. The tightening mechanism may allow the connecting mechanism to pull on the at least two pads, which in turn, may pull on the skin attached to the at least two pads. The device may allow for the stretching of skin in order to immobilize veins while not encroaching on a sterile field.

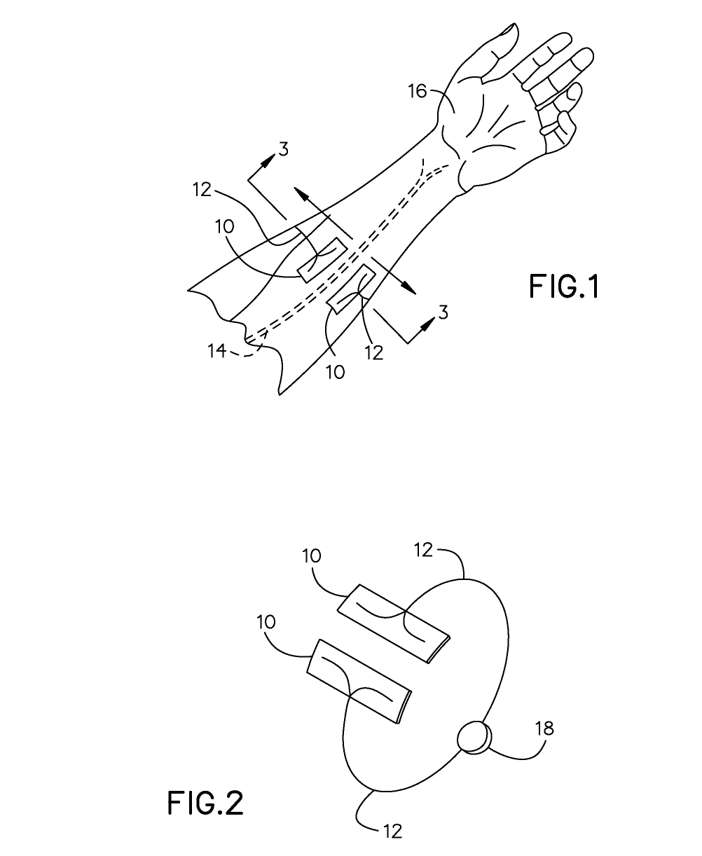

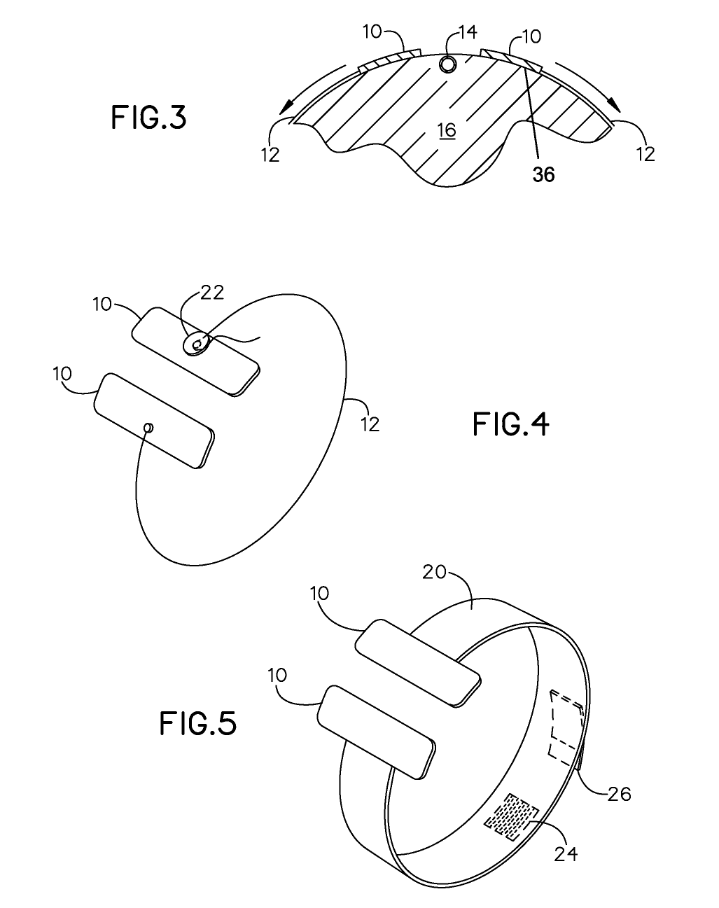

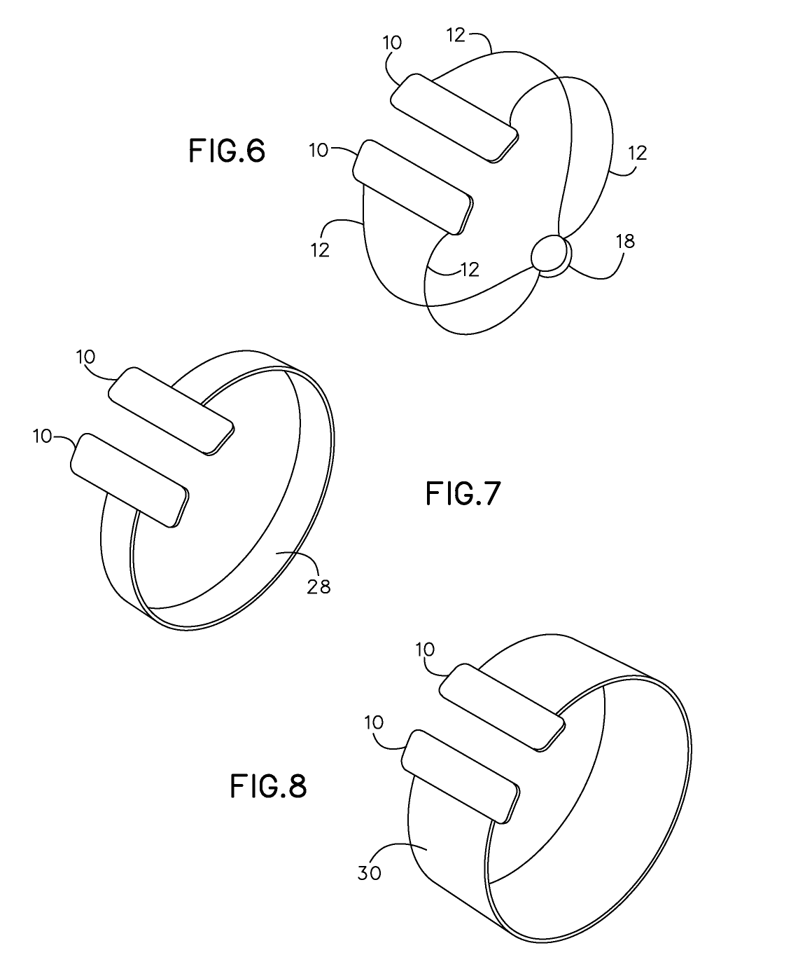

As is illustrated in FIGS. 1 through 8, the phlebotomy aid device may include at least two pads 10 having a body side 36. In certain embodiments, the at least two pads 10 may have adhesive attached to the body side 36 of the pads 10. A connecting mechanism 32 may connect to the at least two pads 10. The connecting mechanism 32 may allow the pads 10 to be connected. The connecting mechanism 32 may be at least one drawstring 12, a tension band 20, a narrow elastic tension band 28, a wide elastic tension band 30 or the like. In certain embodiments, a tightening mechanism 34 may be attached to the connecting mechanism 32 to pull the at least two pads 10 when in use. The tightening mechanism 34 may tighten the connecting mechanism 32 so that the at least two pads 10 may be pulled away from the other/s. The tightening mechanism 34 may be a tightening knob 18, a lash ring 22, a fastener, or the like. When the at least two pads 10 may be attached to a surface, the tightening mechanism 34 may pull the at least two pads 10, which may pull at the surface the at least two pads 10 may be attached to, such as skin of a patient 16. The tightening mechanism 34 may keep the position of the at least one pads 10 so that the skin may stay taut. This position may allow for a vein 14 to be observed better than if the skin was not taut.

A method of using the phlebotomy aid device may include the following. In certain embodiments, the at least two pads 10 may be various shapes such as, but not limited to, rectangular, oval, round, triangular, or the like. The body side 36 of the at least two pads 10 may attach to the skin of the patient 16 by an adhesive or the like. Each pad 10 may be placed on either side of the vein 14 along a patient 16. The at least two pads 10 may be repositioned. In certain embodiments, the drawstring 12 may be attached to each pad 10. Along the drawstring 12 may be a tightening knob 18. The tightening knob 18 may allow the drawstring 12 to be pulled tighter around patient 16. By tightening the drawstring 12, the pads 10 may be pulled, which in turn allows the skin of the patient 16 to become taut. The tightening knob 18 may allow the drawstring and pads 10 to be tightened and kept in place. By tightening the skin of the patient 16 without covering the area of the vein 14, the phlebotomist or the like may be able to have access to the vein 14 and perform what is required without obstruction.

In certain embodiments, an additional drawstring 12 may be attached to the pads 10 and to the tightening knob 18. The additional drawstring 12 may help in distributing the pull throughout the pads 10 to balance the distribution of torque on the pads 10. In alternate embodiments, the tightening mechanism may be the lash ring 22. In certain embodiments, the connecting mechanism 32 may be the tension band 20. The tension band 20 may be tightened around the patient 16 by a tightening mechanism such as a fastener such as a hook fastener 24 and loop fastener 26 or the like. In certain embodiments, the narrow elastic tension band 28, the wide elastic tension band 30 or the like may be used as the connecting mechanism 32. The elastic may provide the additional tightening to help make the skin of the patient taut. In certain embodiments the adhesive may be medical grade pressure sensitive adhesive. In certain embodiments, a cover may removably attach to the adhesive until the device is ready for use.

In certain embodiments, the present invention may be fully adjustable and may gently and effectively provide tension on the skin of a patient 16 to allow easier targeting of the vein 14. The phlebotomy aid device may also enable the phlebotomist to keep both hands free for easier manipulation of the needle and enhanced ability to secure the intravenous catheter or the like. The use of the phlebotomy aid device may help the user with difficult veins 14. The phlebotomy aid device may save the patient 16 multiple injections and reduce costs for the health care professional.

It should be understood, of course, that the foregoing relates to exemplary embodiments of the invention and that modifications may be made without departing from the spirit and scope of the invention as set forth in the following claims.

What is claimed is:

1. A phlebotomy aid device comprising:
at least two pads each having a body side;
an adhesive attached to the body side of the at least two pads; and
a connecting mechanism that connects to the at least two pads.

2. The phlebotomy aid device of claim 1, wherein the connecting mechanism is a drawstring.

3. The phlebotomy aid device of claim 1, wherein the connecting mechanism is a band.

4. The phlebotomy aid device of claim 1, further comprising a tightening mechanism attached to the connecting mechanism.

5. The phlebotomy aid device of claim 4, wherein the tightening mechanism is a tightening knob.

6. The phlebotomy aid device of claim 4, wherein the tightening mechanism is a lash ring.

7. The phlebotomy aid device of claim 4, wherein the tightening mechanism is a fastener.

8. The phlebotomy aid device of claim 1, further comprising a protective covering removably attached to the adhesive.

9. A method for preparing a vein of a patient for a phlebotomy procedure comprising:
attaching an adhesive attached to a body side of at least two pads to a section of skin of the patient, wherein the at least two pads are positioned on either side of the vein, wherein the at least two pads are connected to a connecting mechanism; and
tightening the connecting mechanism, wherein the connecting mechanism then pulls on the at least two pads, wherein the at least two pads pull on the skin providing a taut surface.

10. The method of claim 9, wherein the tightening of the connecting mechanism is by a tightening mechanism.
